(19) United States
(12) Patent Application Publication
(10) Pub. No.: US 2022/0401163 A1
(43) Pub. Date: Dec. 22, 2022

(54) MESH INTRODUCTION CARTRIDGES AND METHODS OF ASSISTED MESH PLACEMENT FOR SURGICAL ROBOTICS

(71) Applicant: Vicarious Surgical Inc., Waltham, MA (US)

(72) Inventors: Sammy Khalifa, Medford, MA (US); Adam Sachs, Somerville, MA (US)

(21) Appl. No.: 17/843,732

(22) Filed: Jun. 17, 2022

Related U.S. Application Data
(60) Provisional application No. 63/211,996, filed on Jun. 17, 2021.

(51) Int. Cl. A61B 34/30

(52) U.S. Cl. CPC A61B 34/30; A61B 2017/00477

(57) ABSTRACT

In an aspect, the present disclosure provides a cartridge that is pre-loaded with a surgical mesh, where the cartridge is configured to be inserted into an internal body cavity of a subject and for the surgical mesh to be deployed while the cartridge is in the internal body cavity of the subject. In another aspect, the present disclosure provides a surgical robotic system comprising a set of sensors embedded thereon, wherein the surgical robotic system is configured to perform a surgical hernia repair procedure with increased consistency.

### RELATED APPLICATIONS

This application claims the benefit of and priority under 35 U.S.C. § 119(e) to U.S. Provisional Patent Application No. 63/211,996, entitled “Mesh Introduction Cartridge and Assisted Mesh Placement for Surgical Robotics,” filed on Jun. 17, 2021. The contents of this application are hereby incorporated by reference in their entirety.

### BACKGROUND OF THE INVENTION

Surgical meshes are used in a number of surgical procedures. Surgical meshes may be provided in a variety of sizes and shapes to treat various sizes and shaped of anatomical defects.

### SUMMARY OF THE INVENTION

The present disclosure provides a device that enables a surgeon to more easily introduce a mesh into a patient for hernia repair. Meshes may be made of a polymeric material (or a biologic or other dissolvable material), and may be introduced via laparoscopic or open surgical technique to be placed over an anatomical defect, for example an abdominal hernia, to help reinforce the tissue and reduce recurrence of future defects. In order to introduce meshes laparoscopically, the mesh may be tightly rolled up, folded, and/or otherwise compressed so that the mesh can be introduced through a trocar or other port. It may be desirable for the mesh to be as tightly rolled, folded, and/or compressed to fit the mesh through as small of a trocar as possible, thereby allowing for easy insertion into a cavity of a subject and reducing the trauma to patient tissue.

Systems, devices, and methods of the present disclosure may have an advantage in reducing recovery time and reducing the occurrence of hernia at the site of the trocar. In some conventional methods, during a medical procedure a surgeon determines a size of surgical mesh to use, and then a surgeon or other medical profession rolls the mesh up tightly keeping the mesh taut so that the mesh can fit through the trocar. This rolling of the mesh to fit through the trocar may require much skill; therefore, surgeons may either struggle to roll the mesh sufficiently tight or choose to employ a larger trocar than may otherwise be necessary. Manual rolling of the mesh to be delivered through the trocar depends on the skill of the surgeon and results may be subject to variability. Further, rolling of the mesh to fit through the trocar can increase the time required for the surgery. In some embodiments, a device of the present disclosure includes a cartridge that may be commercially available and acquired by the surgeon or hospital, and the cartridge is pre-loaded with a tightly rolled, folded, and/or compressed surgical mesh (e.g., a hernia repair mesh) that can subsequently be deployed once introduced through a smaller trocar into the abdomen of the patient. Devices of the present disclosure may enable surgeons to introduce meshes through smaller trocars and add consistency to procedure techniques.

In an aspect, the present disclosure provides a cartridge for storing and delivering a surgical mesh to an internal body cavity during a surgical procedure. The cartridge includes an elongate body defining a cavity and has a first end configured to be a leading end during delivery of the cartridge to the internal body cavity and a second end configured to be a trailing end during delivery of the cartridge to the internal body cavity, one or both of the leading end and the trailing end having an opening therein, a cross-sectional, width, height or diameter of the elongate body being suitable for delivery during the surgical procedure. At least one surgical mesh is at least partially disposed within the cavity.

In some embodiments, the leading end is tapered. In some embodiments, leading end is rounded. In some embodiments, the elongate body includes at least one engagement element on an outer surface or as a portion of the outer surface of the elongate body disposed proximate to the trailing end. In further embodiments, the at least one engagement element is one of textured surface, a protuberance, a handle, or a shape configured to facilitate engagement with a surgical tool.

In some embodiments, the surgical mesh is rolled, folded, or rolled and folded when disposed at least partially within the cavity. In some embodiments, the cartridge further includes a mesh removal element at least partially disposed within the cavity such that withdrawal of the mesh removal element from the cavity initiates removal of the surgical mesh from the cavity. In further embodiments, the mesh removal element comprises one or more of a tab, a string, or an elongate strip. In some embodiments, the mesh removal element is affixed to or is a part of the at least one surgical mesh. In some embodiments, the mesh removal element extends from the leading end of the elongate body. In some embodiments, the mesh removal element extends from the trailing end of the elongate body.

In another aspect, the present disclosure provides a system for storing and delivering at least one surgical mesh to an internal body cavity during a surgical procedure. The system includes the cartridge of the present disclosure, wherein the elongate body has a first opening in the leading end and a second opening in the trailing end, and a deployment element including a distal end disposed in a trailing end of the elongate body and an elongate portion connected with the distal end of the deployment element and extending through the second opening and out from the trailing end of the elongate body, the distal end of the deployment element configured to push the at least one surgical mesh out of the elongate body through the first opening when the distal end of the deployment element is axially displaced relative to the elongate body due to an axial force exerted on a proximal end of the deployment element.

In some embodiments, the distal end of the deployment element has a shape corresponding to a plunger and at least some of the elongate portion of the deployment element has a shape corresponding to a plunger rod. In some embodiments, the system further includes a shaft connected with, affixed to, or integral with the trailing end of the cartridge, the shaft defining a lumen connected to the second opening in the trailing end of the elongate body with the elongate portion of the deployment element extending through the lumen of the shaft. In some embodiments, the system further includes a handle connected to, affixed to, or integral with the proximal end of the shaft, the handle defining an opening connecting with the lumen of the shaft, with the elongate portion of the deployment element extending through the opening of the handle.

In another aspect, the present disclosure provides a method of delivering at least one surgical mesh to an internal body cavity of a subject during a surgical procedure. The method includes delivering a cartridge into the internal body cavity via a channel during the surgical procedure. The cartridge includes an elongate body defining a cavity and has a first end that is a leading end during delivery via the channel and a second end that is a trailing end during delivery via the channel. The at least one surgical mesh is at least partially disposed within the cavity. The method includes retrieving the at least one surgical mesh from the cavity through the leading end or the trailing end of the elongate body or deploying the at least one surgical mesh from the cavity via the leading end or the trailing end of the elongate body.

In some embodiments, delivering the cartridge includes grasping the elongate body with a surgical tool. In further embodiments, the elongate body includes at least one engagement element on an outer surface of the elongate body disposed proximate to the trailing end and a surgical tool grasps the elongate body at the at least one engagement element. In further embodiments, the at least one engagement element is one of textured surface, a protuberance, a handle, or a shape configured to facilitate engagement with the surgical tool.

In some embodiments, retrieving the at least one surgical mesh from the cavity through the leading end or the trailing end of the elongate body includes exerting a force on a mesh removal element that is at least partially disposed within the cavity such that withdrawal of the mesh removal element from the cavity initiates removal of the at least one surgical mesh from the cavity. In further embodiments, the mesh removal element is connected to, affixed to, or integral with the at least one surgical mesh. In some embodiments, the mesh removal element comprises one or more of a tab, a string, or an elongate strip.

In some embodiments, retrieving the at least one surgical mesh includes withdrawing the at least one surgical mesh from the trailing end of the elongate body. In some embodiments, retrieving the at least one surgical mesh includes withdrawing the at least one surgical mesh from the leading end of the elongate body. In some embodiments, retrieving the at least one surgical mesh includes employing an end effector of a robotic arm to withdraw the at least one surgical mesh from the cavity.

In some embodiments, the at least one surgical mesh is deployed from the cavity via the leading end. In further embodiments, deploying the at least one surgical mesh from the cavity via the leading end includes displacing a proximal end of a deployment element relative to the elongate body, the deployment element including a distal end disposed in the cavity of the elongate body and including an elongate portion extending out of the trailing end of the elongate body, the displacement of the proximal end of the deployment element relative to the elongate body pushing the surgical mesh out of the leading end of the elongate body.

In another aspect, the present disclosure provides a method of selecting a surgical mesh and delivering the surgical mesh to an internal body cavity during a surgical procedure. The method includes measuring at least one dimension of an anatomical defect within the internal body cavity during the surgical procedure; determining a cartridge holding a surgical mesh to be selected from a plurality of cartridges based, at least in part, on the measurement of at least one dimension of the anatomical defect; delivering the selected cartridge to the internal body cavity during the surgical procedure; and retrieving the surgical mesh from the cavity through the leading end or the trailing end of the elongate body or deploying the surgical mesh from the leading end or the trailing end of the elongate body. Each cartridge in the plurality includes an elongate body defining a cavity and has a first end configured to be a leading end during delivery of the cartridge to the internal body cavity and a second end configured to be a trailing end during delivery of the cartridge to the internal body cavity. A surgical mesh is at least partially disposed within the cavity, the mesh configured to be secured to a surgical site over an anatomical defect, wherein each cartridge in the plurality of cartridges holds a surgical mesh having a different length, width, or diameter than that held by other cartridges in the plurality of cartridges.

In some embodiments, measuring at least one dimension of the anatomical defect includes touching one or more edges of the anatomical defect with a distal end of a robotic arm of a surgical robotic system. In further embodiments, the surgical robotic system determines the cartridge to be selected based on the measure at least one dimension of the anatomic defect. In further embodiments, the surgical robotic system displays information regarding the determined cartridge to be selected via a user interface. In some embodiments, measuring at least one dimension of the anatomical defect comprises obtaining at least one image from a camera system positioned in the internal body cavity. In further embodiments, measuring at least one dimension of the anatomical defect further includes determining the at least one dimension of the anatomical defect from the obtained at least one image of the camera system. In some embodiments, the measuring of at least one dimension of an anatomical defect within the internal body cavity during the surgical procedure and the determining the cartridge to be selected from the plurality of cartridges based, at least in part, on the measurement of at least one dimension of the anatomical defect is conducted by a surgical imaging or robotic system.

Further, the present disclosure provides systems, devices, and methods for assisted mesh placement for surgical robotics. Hernia repair may be a procedure that comprises closing a wound or defect in tissue, such as muscle, of a subject. A common surgical repair technique may include utilizing a surgical mesh placed over the hernia to reinforce the tissues surrounding the hernia. Meshes may be made of a polymeric material, or a biologic or dissolvable material. Due to variability in the ways surgeons place meshes, there may be significant variability in patient outcomes. Systems, devices, and methods of the present disclosure may use sensors embedded on a surgical robotic system to help increase consistency of hernia repair procedures, and may be combined with use of a comprehensive database that includes specific measurable parameters as well as patient outcomes.

Systems, devices, and methods for assisted mesh placement for surgical robotics may advantageously bring together and analyze data that is available to surgical robotics platforms with patient outcomes to improve the outcomes of hernia repair procedures. Further, the system may be intelligent and adaptable to different conditions, while still allowing increased consistency of procedures performed.

In another aspect, the present disclosure provides a surgical robotic system comprising a set of sensors embedded thereon, wherein the surgical robotic system is configured to perform a surgical hernia repair procedure with increased consistency.

In another aspect, the present disclosure provides a method for using a set of sensors embedded on a surgical robotic system to perform a surgical hernia repair procedure with increased consistency.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings, which are not to scale.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6 schematically depicts a cross-sectional view and an open end view of a mesh rolled up inside a cavity of an elongate body of a cartridge in accordance with some embodiments.

FIG. 7A schematically depicts a side cross-sectional view a cartridge having a tapered leading end in accordance with some embodiments.

FIG. 7B schematically depicts a side cross-sectional view of a cartridge having a rounded leading end in accordance with some embodiments.

FIG. 8A schematically depicts a side cross-sectional view of a cartridge having an open leading end in accordance with some embodiments.

FIG. 8B schematically depicts a side cross-sectional view of a cartridge having an open trailing end in accordance with some embodiments.

FIG. 8C schematically depicts a side cross-sectional view of a cartridge having an open leading end and an open trailing end in accordance with some embodiments.

FIG. 9A schematically depicts a side cross-sectional view of a cartridge having a textured surface portion in accordance with some embodiments.

FIG. 9B schematically depicts a side cross-sectional view of a cartridge having a protuberance in accordance with some embodiments.

FIG. 9C schematically depicts a side cross-sectional view of a cartridge having a handle in accordance with some embodiments.

FIG. 10A schematically depicts a side cross-sectional view of a cartridge and a mesh removal element partially disposed within a cavity of the cartridge and protruding from the cavity in accordance with some embodiments.

FIG. 10B schematically depicts a side cross-sectional view of a cartridge and a different mesh removal element partially disposed within a cavity of the cartridge and protruding from the cavity in accordance with some embodiments.

FIG. 10C schematically depicts a side cross-sectional view of a cartridge and a deployment element partially disposed within a cavity of the cartridge and partially protruding from the cavity in accordance with some embodiments.

FIG. 10D schematically depicts a side cross-sectional view of a cartridge, a deployment element partially disposed within a cavity of the cartridge, and a shaft connected with an elongate body of the cartridge in accordance with some embodiments.

FIG. 11A schematically depicts a side cross-sectional view of a laparoscopic assist tool inserting a cartridge through a trocar in accordance with some embodiments.

FIG. 11B schematically depicts a side view of a first laparoscopic assist tool inserting a cartridge through a trocar and a second laparoscopic tool removing a surgical mesh from the cartridge in accordance with some embodiments.

FIG. 11C schematically depicts a side view of a first laparoscopic assist tool inserting a cartridge through a trocar and a second laparoscopic tool removing a surgical mesh having a protruding end from the cartridge in accordance with some embodiments.

FIG. 12 schematically depicts a side view of inserting a cartridge through a trocar with the assistance of a sleeve in accordance with some embodiments.

FIG. 13A schematically depicts a side view of deploying a surgical mesh from a leading end of a cartridge with the assistance of an elongate element in accordance with some embodiments.

FIG. 13B schematically depicts a side view of deploying a surgical mesh from a leading end of a cartridge in accordance with some embodiments.

FIG. 14 depicts an image of a mesh unrolled and fixed to the inside of a subject's abdomen during an intraperitoneal onlay mesh repair (IPOM) procedure in accordance with some embodiments.

FIG. 15 schematically depicts a process including guiding a surgeon during surgery and selection of pre-filled cartridges in accordance with some embodiments.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise.

The present disclosure describes devices, systems, and methods that may be employed during surgical procedures. In some embodiments, the surgical procedures are minimally invasive surgeries. The term “minimally invasive surgery” may be understood to indicate a surgical procedure performed through one or more small incisions, typically with laparoscopic instruments and cameras.

Surgical meshes are used in a number of surgical procedures. Surgical meshes may be provided in a variety of sizes and shapes to treat a variety of sizes and shapes of anatomical defects in some embodiments. Surgical meshes used to repair an anatomical defect may be introduced into a body via laparoscopic or open surgical technique, placed over an anatomical defect, for example an abdominal hernia, and secured to tissue to help reinforce the tissue and reduce recurrence of future defects. Such meshes may be made of a polymeric, material, a biologic material, a dissolvable material, any combination of the aforementioned, or any other suitable material. In order to introduce a surgical mesh laparoscopically, the mesh must be tightly rolled up, folded, and/or otherwise compressed so that the mesh can be introduced through a trocar or other port. It may be desirable for the mesh to be more tightly rolled, folded, and/or compressed to fit the mesh through as small of a trocar as possible, thereby allowing for easy insertion into a cavity of a subject and reducing the trauma to patient tissue.

In some conventional methods, during a medical procedure, a surgeon determines a size of surgical mesh to use, and then the surgeon or other medical professional selects a mesh of the appropriate size or cuts a mesh to the appropriate size, and then rolls the mesh up tightly keeping the mesh taut so that the mesh can fit through the trocar. This rolling of the mesh to fit through the trocar involves difficulty and requires skill. Surgeons may either struggle to roll the mesh sufficiently tight or choose to employ a larger trocar than may otherwise be necessary. Manual rolling of the mesh to be delivered through the trocar depends on the skill of the surgeon and results may be subject to variability from surgeon to surgeon and day to day. Further, the time required rolling of the mesh to fit through the trocar could increase the time required for the surgery.

The present disclosure provides systems, devices, and methods that may enable a surgeon to more easily and consistently introduce a mesh into a patient for repair of an anatomic defect (e.g., hernia repair). Systems, devices, and methods of the present disclosure may have an advantage in reducing recovery time and reducing the occurrence of hernia at the site of the trocar used to introduce the surgical mesh.

In some embodiments, a device of the present disclosure includes a cartridge that may be commercially available and acquired by the surgeon or hospital, and the cartridge is pre-loaded with a tightly rolled, folded, and/or compressed surgical mesh (e.g., a hernia repair mesh), that can subsequently be deployed once introduced through a smaller trocar into the abdomen of the patient than could be achieved by a surgeon hand rolling a surgical mesh. Devices, systems, and methods of the present disclosure may enable surgeons to introduce meshes through smaller trocars and increase consistency of procedure techniques.

In an aspect, the present disclosure provides a device including a cartridge that is pre-loaded with a surgical mesh, where the surgical repair mesh is configured to be inserted into a cavity of a subject and to be deployed or removed from the cartridge while the cartridge is in the body cavity of the subject.

In another aspect, the present disclosure provides a method for using a device comprising a cartridge that is pre-loaded with a surgical mesh, the method including inserting the surgical mesh into a cavity of a subject, and deploying the surgical mesh while the cartridge is inserted in the cavity of the subject.

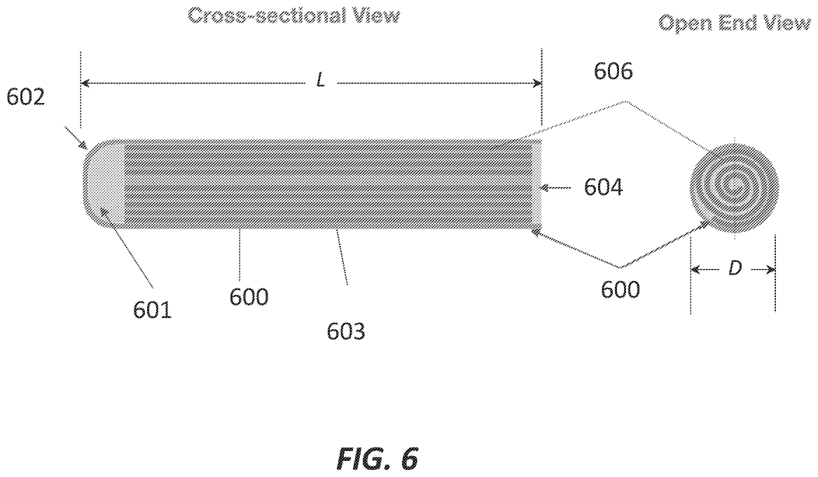

Prior to addressing embodiments of devices, systems and methods for delivery of a surgical mesh in detail with respect to FIGS. 6-15, a description is provided of example surgical robotics systems and robotic assemblies in which some embodiments may be employed or implemented. One of ordinary skill in the art in view of the present disclosure will appreciate that embodiments can be employed or implemented using different types of surgical instruments and surgical systems, and in different types of surgical procedures, and are not limited to use with surgical robotic systems or surgical robotic assemblies.

### Surgical Robotic Systems

Some embodiments may be employed with a surgical robotic system. A system for robotic surgery may include a robotic subsystem that includes a surgical robotic unit that can be inserted into a patient via a trocar through a single incision point or site. The robotic unit is small enough to be deployed in vivo at the surgical site and is sufficiently maneuverable when inserted to be able to move within the body to perform various surgical procedures at multiple different points or sites. The surgical robotic unit includes multiple separate robotic arms that are deployable within the patient along different or separate axes. Further, a surgical camera assembly can also be deployed along a separate axis. Thus, the surgical robotic unit employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor unit and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

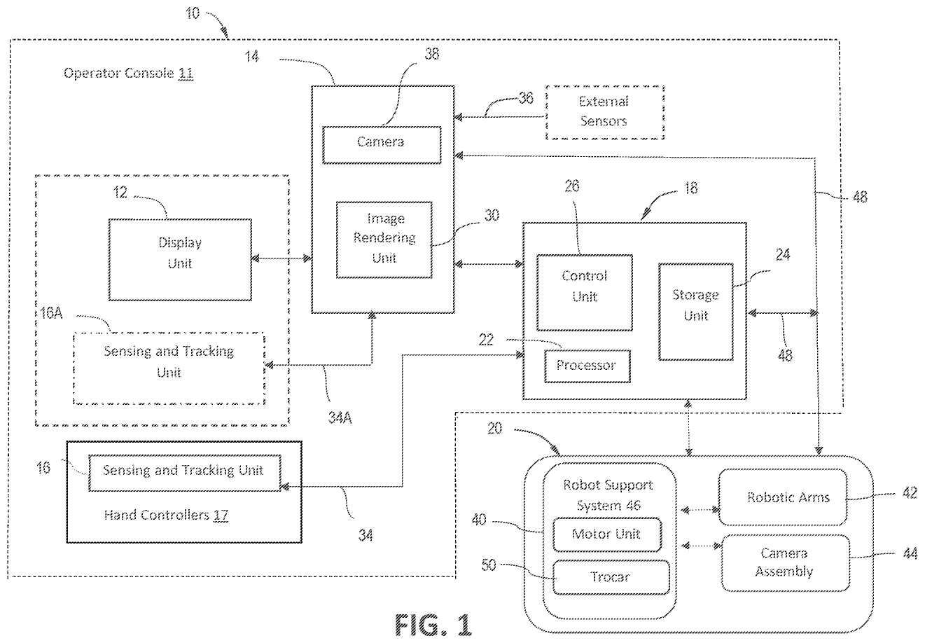

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display device or unit 12, an image computing unit 14, which may be a virtual reality (VR) computing unit, hand controllers 17 having a sensing and tracking unit 16, and a computing unit 18.

The display unit 12 can be any selected type of display for displaying information, images or video generated by the image computing unit 14, the computing unit 18, and/or the robotic subsystem 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 of the robotic subsystem 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuity, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and robotic arms 42 of the robotic subsystem 20. The tracking and position data 34 generated by the sensing and tracking unit 16 can be conveyed to the computing unit 18 for processing by at least one processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage unit 24. The tracking and position data 34A can also be used by the control unit 26, which in response can generate control signals for controlling movement of the robotic arms 42 and/or the camera assembly 44. For example, the control unit 26 can change a position and/or an orientation of at least a portion of the camera assembly 44, of at least a portion of the robotic arms 42, or both. In some embodiments, the control unit 26 can also adjust the pan and tilt of the camera assembly 44 to follow the movement of the operator's head.

The robotic subsystem 20 can include a robot support system (RSS) 46 having a motor unit 40 and a trocar 50, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50. The RSS 46 can further include a support member that supports the motor unit 40 coupled to a distal end thereof. The motor unit 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor unit 40 that is coupled to the robotic subsystem 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robotic arms 42 and the cameras assembly 44 separately or together. The motor unit 40 can also provide mechanical power, electrical power, mechanical communication, and electrical communication to the robotic arms 42, the camera assembly 44, and/or other components of the RSS 46 and robotic subsystem 20. The motor unit 40 can be controlled by the computing unit 18. The motor unit 40 can thus generate signals for controlling one or more motors that in turn can control and drive the robotic arms 42, including for example the position and orientation of each articulating joint of each robotic arm, as well as the camera assembly 44. The motor unit 40 can further provide for a translational or linear degree of freedom that is first utilized to insert and remove each component of the robotic subsystem 20 through the trocar 50. The motor unit 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. The robotic subsystem 20 can be supported by the trocar with multiple degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robotic arms 42, the camera assembly 44, and the like), and for generating control signals in response thereto. The motor unit 40 can also include a storage element for storing data.

The robotic arms 42 can be controlled to follow the scaled-down movement or motion of the operator's arms and/or hands as sensed by the associated sensors. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms of the robotic arms 42 may follow movement of the arms of the operator in some modes of control, the robotic shoulders are fixed in position in such modes of control. In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed.

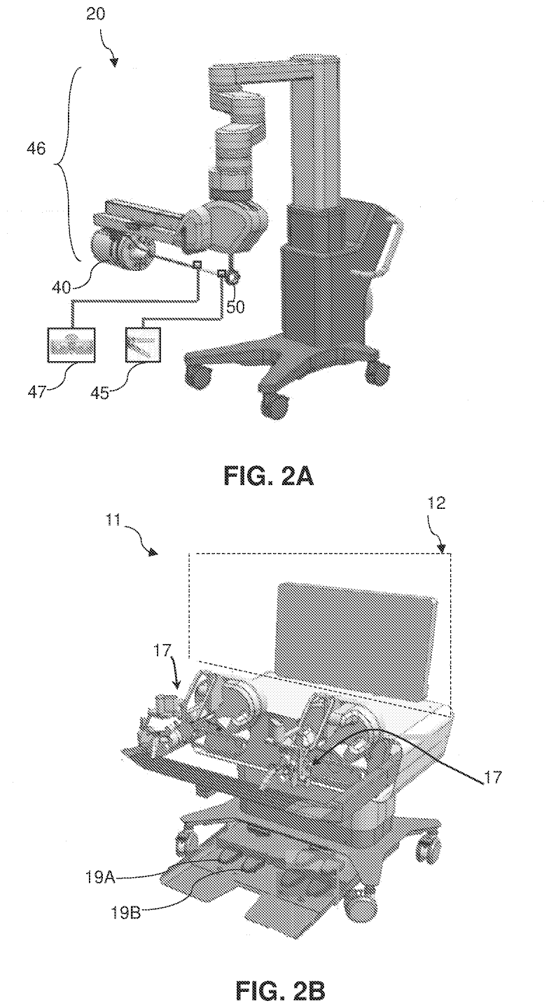

FIG. 2A depicts an example robotic assembly 20 of a surgical robotic system 10 incorporated into or mounted onto a mobile patient cart in accordance with some embodiments. In some embodiments, the robotic assembly 20 includes the RSS 46, which, in turn includes the motor unit 40, the robotic arm assembly 42 having end-effectors 45, the camera assembly 44 having one or more cameras 47, and may also include the trocar 50.

FIG. 2B depicts an example of an operator console 11 of the surgical robotic system 10 of the present disclosure in accordance with some embodiments. The operator console 11 includes a display unit 12, hand controllers 17, and may also include one or more additional controllers (e.g., foot pedals or switches) for control of the robotic arms 42, for control of the camera assembly 44, and for control of other aspects of the system.

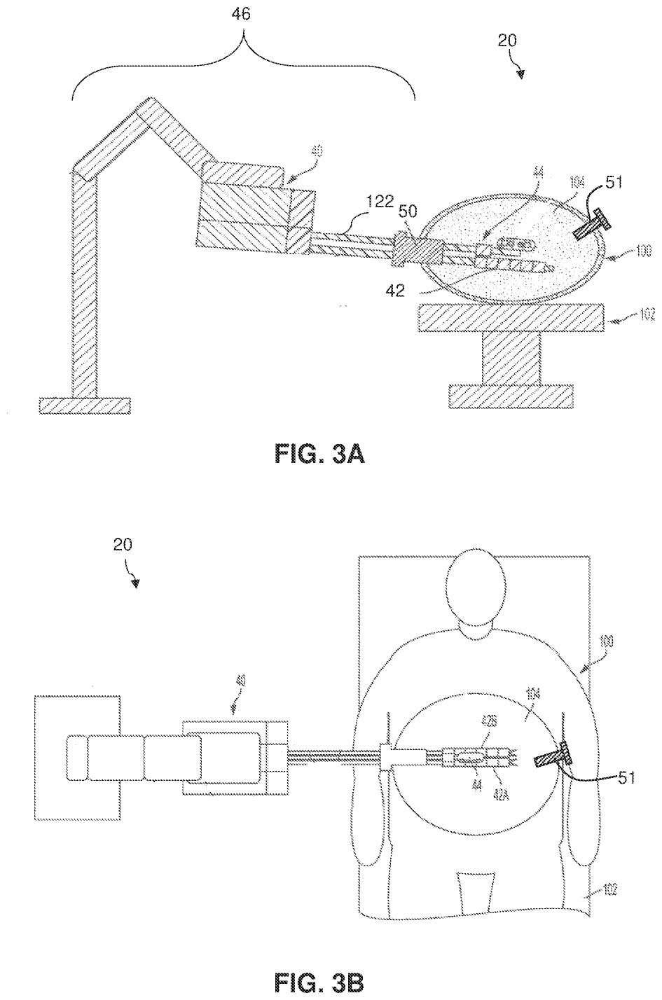

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. The robotic assembly 20 can be coupled to the motor unit 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

During a surgery, a second trocar 52 may be positioned at a different location on the subject from that of the first trocar 60 and may be employed for delivering a cartridge carrying a surgical mesh into the internal cavity of the patient in accordance with some embodiments. In some embodiments, the second trocar 52 has a smaller inner diameter than that of the first trocar 50.

### Robotic Assembly Control

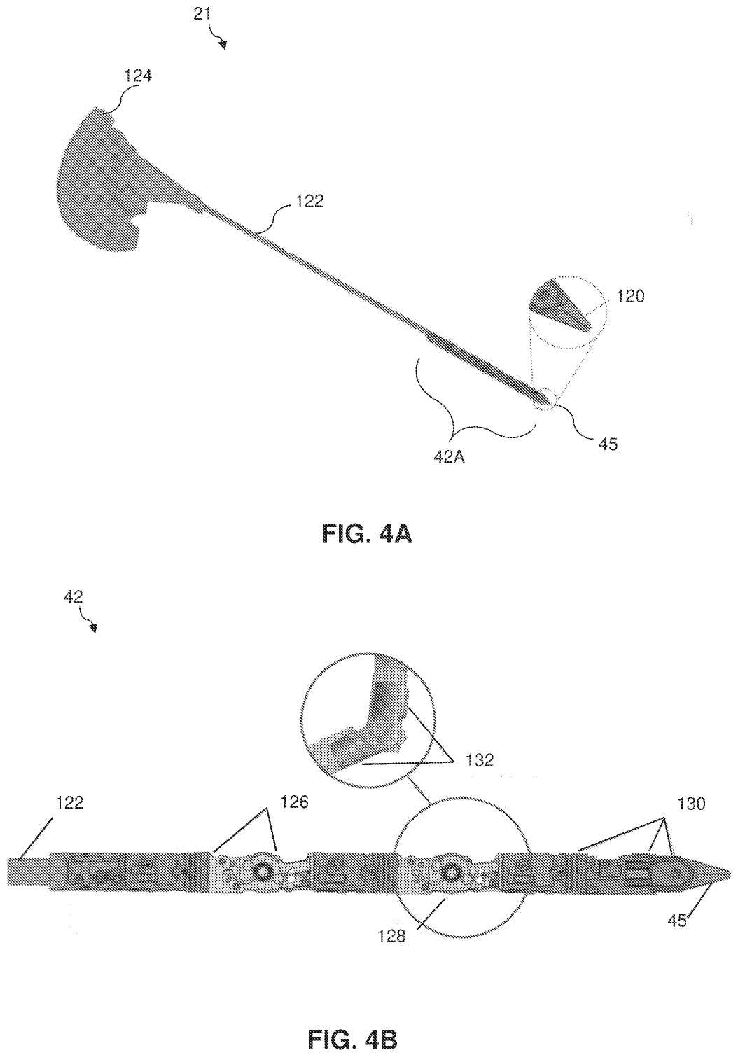

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor unit 40 (as shown in FIGS. 1 and 2A). At least a portion of the shaft 122 can be external to the internal cavity 104 (as shown in FIGS. 3A and 3B). At least a portion of the shaft 122 can be inserted into the internal cavity 10 (as shown in FIGS. 3A and 3B).

FIG. 4B is a side view of the robotic arm assembly 42. The robotic arm assembly 42 includes a virtual shoulder 126, a virtual elbow 128 having capacitive proximity sensors 132, a virtual wrist 130, and the end-effector 45. The virtual shoulder 126, the virtual elbow 128, the virtual wrist 130 can include a series of hinge and rotary joints to provide each arm with positionable, seven degrees of freedom, along with one additional grasping degree of freedom for the end-effector 45.

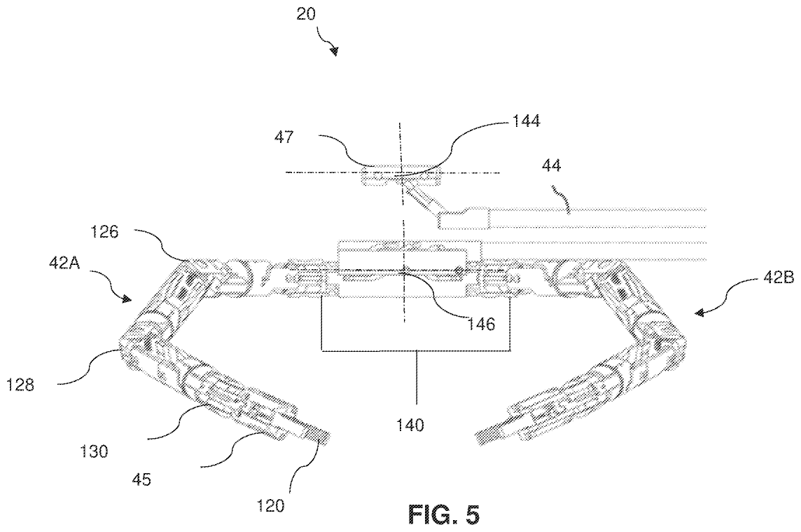

FIG. 5 illustrates a perspective front view an internal portion of the robotic assembly 20. The robotic assembly 20 includes a first robotic arm 42A and a second robotic arm 42B. The two robotic arms 42A and 42B can define a virtual chest 140 of the robotic assembly 20. The virtual chest 140 can be defined by a chest plane extending between a first pivot point 142A of a most proximal joint of the first robotic arm 42A, a second pivot point 142B of a most proximal joint of the second robotic arm 42B, and a camera imaging center point 144 of the camera(s) 47. A pivot center 146 of the virtual chest 140 lies midway along a line segment in the chest plane connecting the first pivot point 144 of the first robotic arm 42A and the second pivot point 142B of the second robotic arm. 42B.

In some embodiments, sensors in one or both of the first robotic arm 42A and the second robotic arm 42B can be used by the system to determine a change in location in three-dimensional space of at least a portion of the robotic arm. In some embodiments, sensors in one of both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Cartridges for Delivering Surgical Mesh

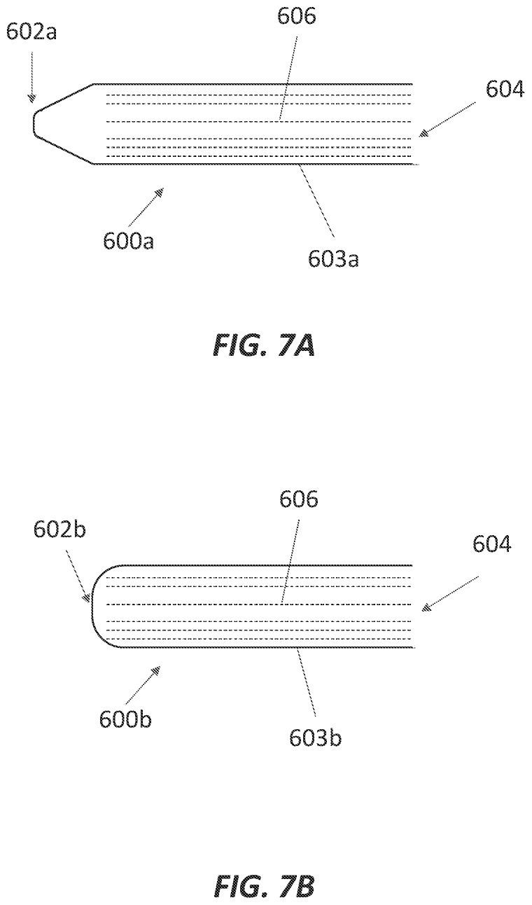

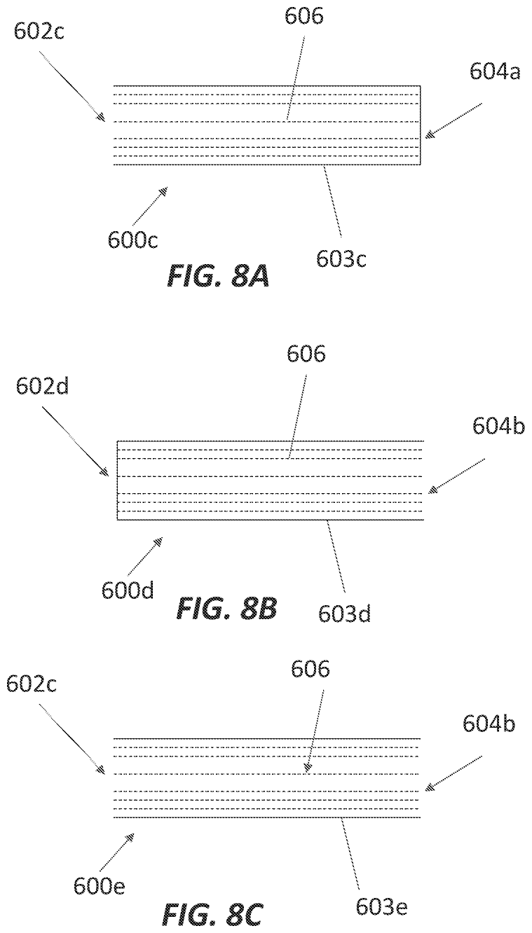

As explained above, some embodiments provide cartridges for delivery of a surgical mesh to an internal body cavity of a subject. FIG. 6 depicts a side cross-sectional view and a rear trailing end view of a cartridge 600 for delivery of a surgical mesh 606 in accordance with some embodiments. The cartridge 600 includes an elongate body 603 defining a cavity 601. In some embodiments, the elongate body may have a shape of, or may be formed in part by, a thin-walled tube. The elongate body 603 has a first end 602 and a second end 604. The first end 602 may be configured to be a leading end during delivery of the cartridge 600 to an internal body cavity and may be referred to as the “leading end” herein. The second end 604 may be configured to be a trailing end during delivery of the cartridge 600 to the internal body cavity and may be referred to as the “trailing end” herein. In some embodiments, one end (e.g., a first end) of the elongate body includes an opening and another end (e.g., a second end) is closed. For example, cartridge 600 includes a closed leading end 602 and an open trailing end 604. See also, e.g., FIGS. 7A, 7B, 8A, and 8B. In some embodiments, both the leading end and the trailing end include an opening (see, e.g., FIG. 8C). In some embodiments, neither the leading end nor the trailing end includes an opening, but the elongate body is configured have an opening formed in one or both ends to remove or deploy the surgical mesh after the cartridge is delivered to the internal body cavity. For example, in some embodiments an end of the elongate body may include perforations, or weakened areas configured to tear or split. In some embodiments, an end of the elongate body may include an opening smaller than a maximum inner diameter of the elongate body. In some embodiments, an end of the elongate body may include an opening smaller than a maximum inner diameter of the elongate body and one or more perforations or weakened areas configured to split or tear near the opening.

In some embodiments, the elongate body includes a polymeric material. In some embodiments, the elongate body includes a plastic material. In some embodiments, the elongate body includes a biogenic material. In some embodiments, the elongate body includes any biocompatible materials including bioabsorable materials. In some embodiments, the elongate body includes any combination of the aforementioned. In some embodiments, the elongate body has coating on at least a portion of one or both of an inner surface (e.g., a surface facing the cavity 601) and an outer surface (e.g., a surface facing away from the cavity 601). In some embodiments, a coating on at least a portion of an inner surface of the elongate body may be different from a coating on at least a portion of an outer surface of the elongate body. In some embodiments, a coating on at least an inner portion of the inner surface may reduce friction between the elongate body 603 and the surgical mesh 603. In some embodiments, a coating or coatings include any biocompatible materials including bioabsorbable materials.

The elongate body 603 may have a cross-sectional, width, height, or diameter D suitable for delivery during the surgical procedure, which may be a minimally invasive surgical (MIS) procedure. The elongate body 603 may have a cross-sectional width, height, or diameter enabling use in a relatively small diameter trocar. For example, in some embodiments, the diameter of the elongate body 603 may range between 3 mm and 35 mm. In some embodiments, the diameter may range between 5 mm and 12 mm. In some embodiments, the diameter may range between 6 mm and 12 mm. In some embodiments, a length L of the elongate body 603 may range between 5 cm to 10 cm.

FIG. 7A schematically depicts a side cross-sectional view of a tapered leading end 602a of an elongate body 603a of a cartridge 600a in accordance with some embodiments. In some embodiments, the leading end 602a is tapered into a blunt or smooth shape, for example, a conical shape or a conical shape with a rounded tip. The tapered end 602a of the elongate body 603a of the cartridge 600a may aid in smooth introduction of the cartridge 600a through the trocar 50.

In some embodiments, a leading end 602b of an elongate body 603a of a cartridge is rounded 602b. FIG. 7B schematically depicts a side cross-sectional view of a rounded leading end 602b of an elongate body 603b of a cartridge 600b in accordance with some embodiments. A rounded shape may aid in smooth introduction of the cartridge 600b into a trocar reduce a likelihood of damage to the internal body cavity by the cartridge 600b. A cross-sectional maximum width, height and/or diameter of the elongate body 603b of the cartridge 600b may be suitable for delivery during a minimally invasive surgical procedure, for example, a MIS hernia repair.

FIG. 8A schematically depicts a side cross-sectional view of a cartridge 600c with an elongate body 603c having an open leading end 602c and a closed trailing end 604a in accordance with some embodiments. FIG. 8B schematically depicts a side cross-sectional view of a cartridge 600d having an elongate body 603d with an open trailing end 604b and a closed leading end 602d in accordance with some embodiments. FIG. 8C schematically depicts a side cross-sectional view of an elongate body 603e of a cartridge 600e having an open leading end 602c and an open trailing end 604b in accordance with some embodiments. In some embodiments, the cartridge is open on one end or both ends. Open ends of the cartridge facilitate retrieval or deployment of the surgical mesh 606, as described in further detail below.

As explained above, a rolled, folded, or rolled and folded surgical mesh 606 may be at least partially disposed in a cavity of an elongate body of a cartridge 60. Upon successful introduction of the cartridge through a trocar or port (e.g., 52 of FIG. 3A or 3b) into an internal body cavity of a subject (e.g., a patient cavity), the surgical mesh 606 may be removed or deployed from the cartridge 600, unrolled, unfolded, unfolded and then unrolled, or unrolled and then unfolded, and then used, for example, as a hernia repair mesh in a hernia repair surgery.

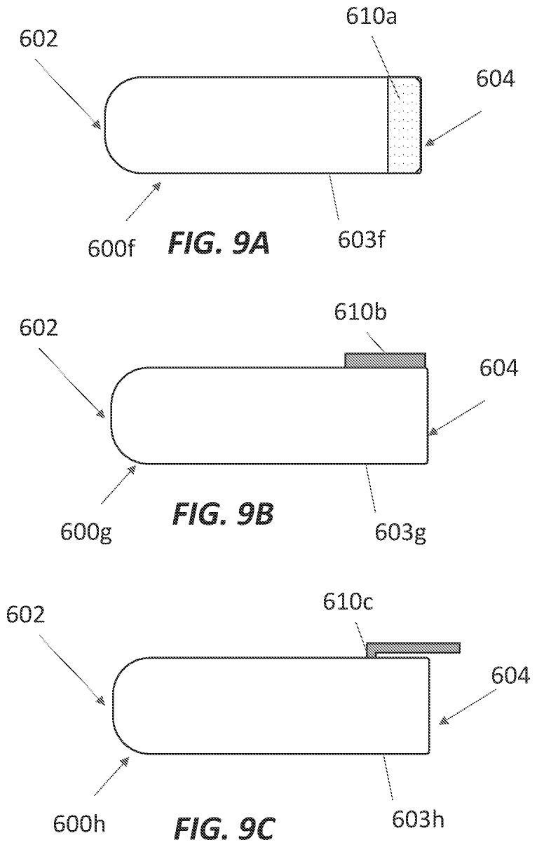

In some embodiments, a cartridge includes are one or more engagement elements on, or as a portion of, the elongate body that aid in fixing or holding the cartridge in place while the surgical mesh is removed. These features may include any of textures, protuberances, or a shape configured to facilitate engagement with a surgical tool (e.g., one or more handles designed to interface with surgical tools such as surgical graspers). FIG. 9A schematically depicts a side view of a cartridge 600f including an elongate body 603f having a textured outer surface portion 610a in accordance with some embodiments. FIG. 9B depicts a side cross-sectional view of a cartridge 600g including an elongate body 603g and a protuberance 610b in accordance with some embodiments. FIG. 9C schematically depicts a side cross-sectional view of a cartridge 600h including an elongate body 603 and a handle 610c in accordance with some embodiments. In some embodiments, the engagement elements or elements 610a, 610b, 610c are disposed proximate to the trailing end 604 of the cartridge 600.

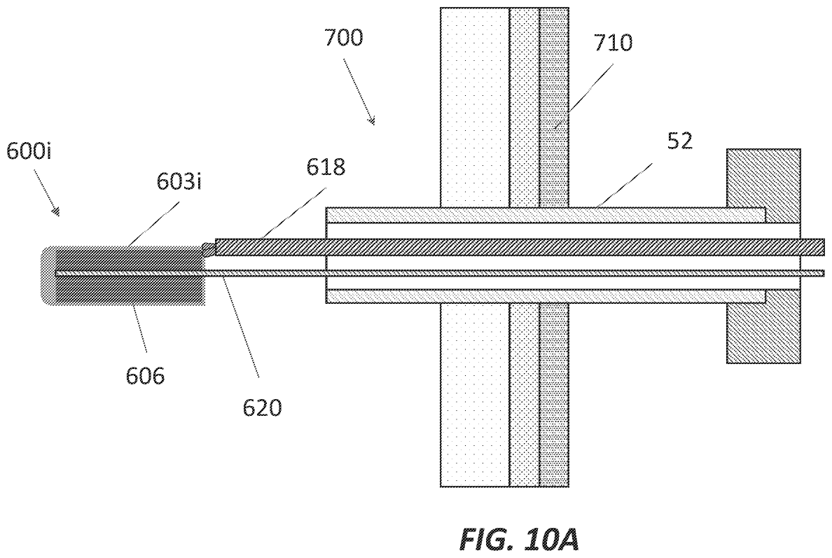

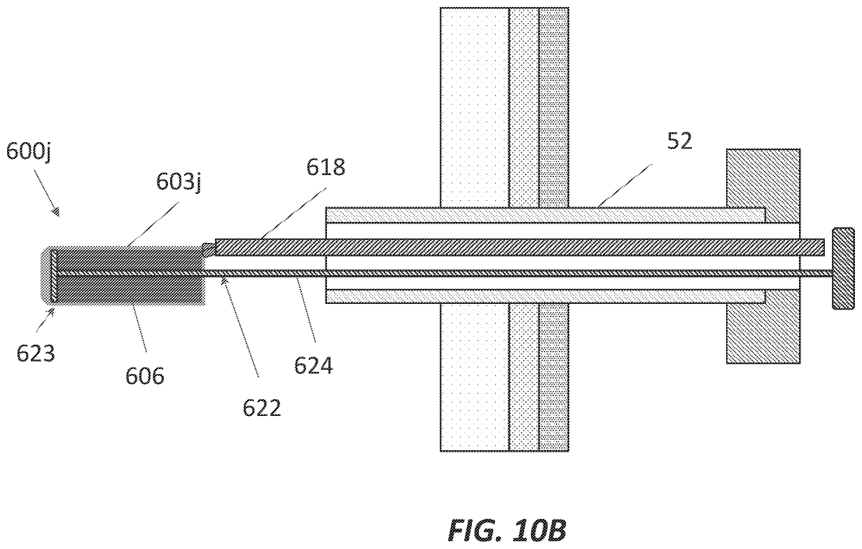

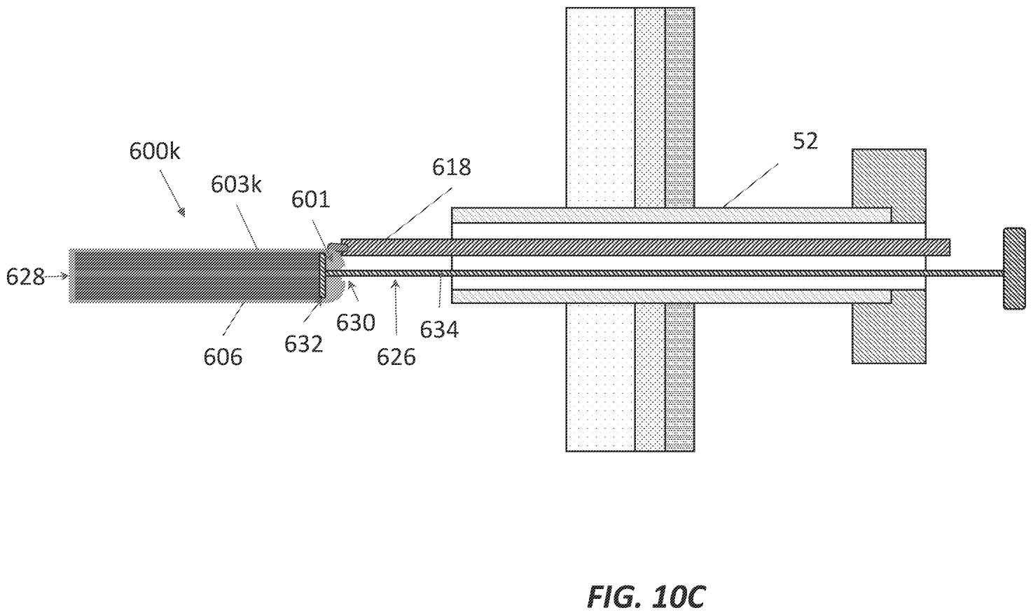

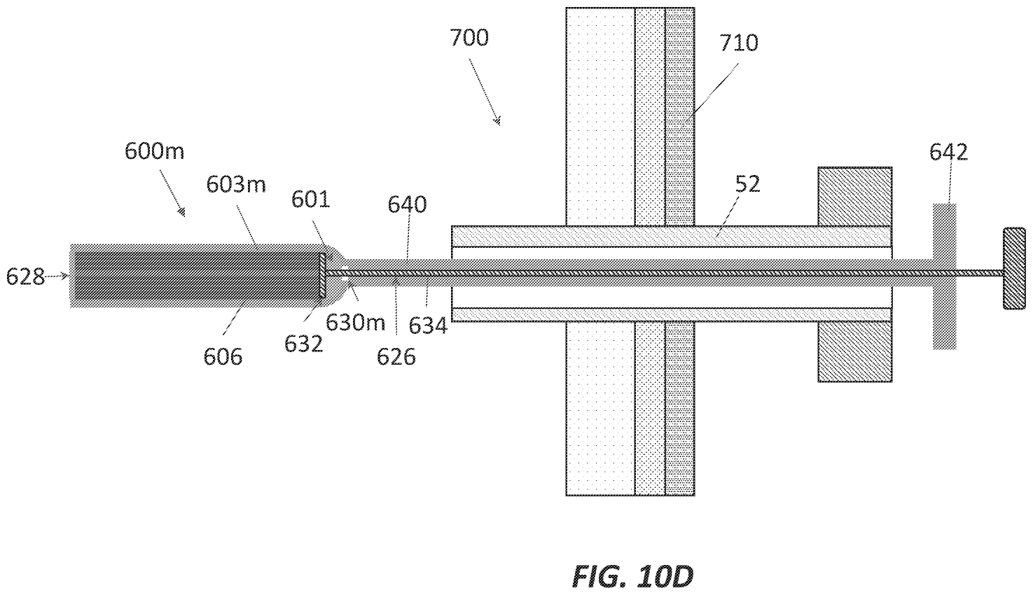

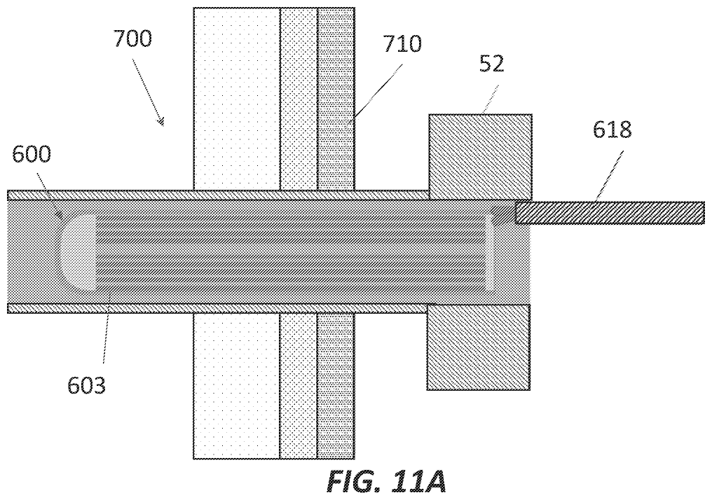

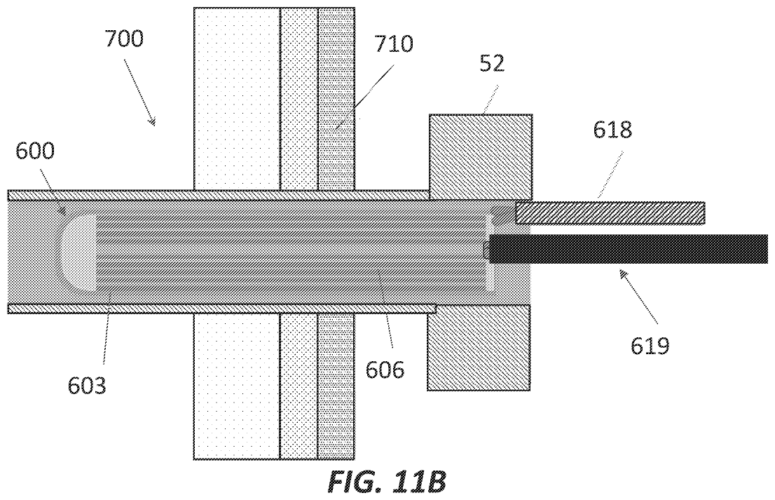

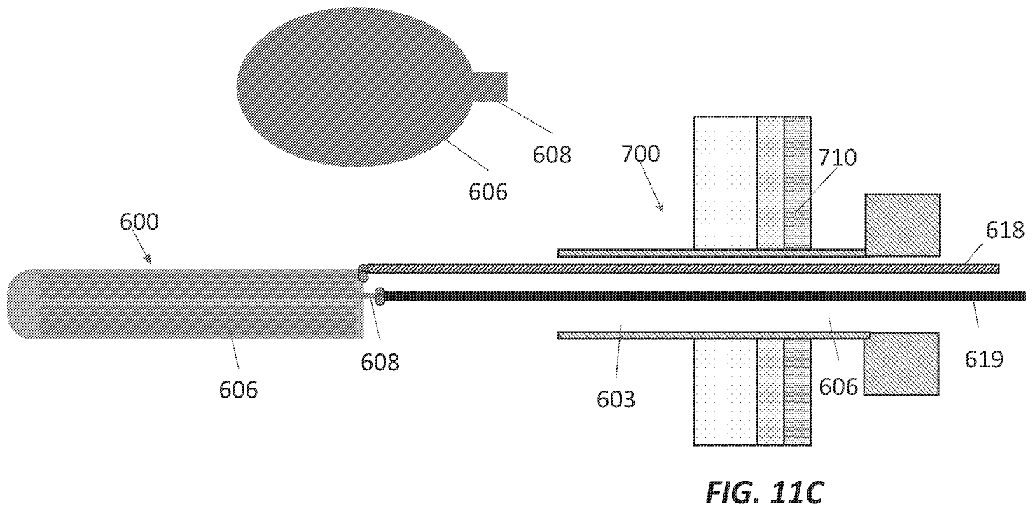

In some embodiments, at least one a mesh removal element, for example, as a string, a tab, or an elongate strip, is at least partially disposed within the cavity of the elongate body with the surgical mesh 606 to aid in removal of the surgical mesh from the cartridge. See e.g., FIGS. 10A-10C, 11C and accompanying description below. Withdrawal of the at least one mesh removal element from the cavity 601 initiates or causes removal of the surgical mesh from the cavity. In some embodiments, the at least one mesh removal element is affixed to or is a part of the surgical mesh. In some embodiments, the at least one mesh removal element extends from the leading end of the elongate body. In other embodiments, the at least one mesh removal element extends from the trailing end of the elongate body.

FIG. 10A schematically depicts a side cross-sectional view of a cartridge 600i being delivered through an abdominal wall 710 via a trocar into an internal body cavity 700 of a subject. The cartridge 600 includes a mesh removal element including a string disposed partially within a cavity 601 of the elongate body 603i of the cartridge 600 and partially protruding from the cavity 601 in accordance with some embodiments. The string 620 may be pulled externally by the surgeon to remove the mesh 606 from the cartridge 600i. In some embodiments, the mesh withdrawal element (e.g., string 620) is attached the surgical mesh 606. In some embodiments, the string 620 is removably attached to the surgical mesh 606, such that the string 620 can be removed or cut off the surgical mesh 606 once the mesh surgical 606 is removed from the cartridge 600i. In some embodiments, the mesh removal element is not attached to the mesh 606, and simply relies on friction between mesh removal element and the mesh 606 to transfer sufficient force to withdraw the mesh 606 due to the externally applied pulling force on the mesh removal element. In some embodiments, at least a portion of the mesh removal element (e.g., string 620) is rolled up, folded, or rolled and folded into the mesh 606 to increase the friction between the mesh removal element (e.g., string 620) and the mesh 606, and improve the ability for the string 620 to pull the mesh 606 out of the cartridge 600i.

FIG. 10B depicts a side view of a cartridge 600j including mesh removal element 622 at least partially disposed within a cavity 601 of an elongate body 603j of the cartridge 600j and partially protruding from the cavity 601 in accordance with some embodiments. In some embodiments, a distal end 623 of the mesh removal element 622 has a larger diameter that at least some other portions of the mesh removal element and is disposed in the cavity distal to at least some of, most of, or all of the surgical mesh 606. In some embodiments, the distal end 623 of the mesh removal element 622 has a shape corresponding to a plunger or a plate. In some embodiments, at least some of the portion 624 of the mesh removal element 622 has a shape corresponding to a rod or a plunger rod.

In some embodiments, a length of the mesh removal element 520, 522 is sufficient for a proximal end of the mesh removal element to extend through and out of a trocar 52 in use. In use, pulling on a proximal end of the mesh removal element 622 displaces it in a proximal direction with respect to the elongate body 603i causing it to push the surgical mesh 606 out of the cavity in accordance with some embodiments. In some embodiments, a surgical tool 618, e.g., an assist laparoscopic tool may be employed to advance a cartridge 600j through the trocar and to hold the elongate body 603i during deployment of the surgical mesh 606.

FIG. 10C depicts a side view of a system for delivery of a surgical mesh including a cartridge 600k and a deployment element 626 at least partially disposed within a cavity 601 of an elongate body 603k of the cartridge 600k and partially protruding from the cavity 601 in accordance with some embodiments. The elongate body has an opening 628 at a leading end and a smaller diameter opening 630 at a trailing end. In some embodiments, a distal end 632 of the deployment element 626 has a larger diameter than at least some other portions of the mesh removal element and is disposed in the cavity 601 between the mostly closed trailing end of the elongate body (e.g., a proximal end of elongate body 603j) and the surgical mesh 606. The distal end 632 may have a shape corresponding to a plate or a bung of a plunger in some embodiments. In some embodiments, at least some of the deployment element has a shape corresponding to a rod or a plunger rod 624.

In some embodiments, a length of the deployment element 626 is sufficient for a proximal end of the deployment element to extend through and out of a trocar 52 in use. In use, pushing on a proximal end of the deployment element 626 displaces the distal end 632 with respect to the elongate body 603k causing the distal end 632 to push the surgical mesh 606 out of the cavity in accordance with some embodiments. In some embodiments, a surgical tool 618, e.g., an assist laparoscopic tool may be employed to advance the cartridge 600k through the trocar and to hold the elongate body 603k during deployment of the surgical mesh 606.

Turning to FIG. 10D, in some embodiments, a system for delivery of a surgical mesh may include a cartridge 600m, a deployment element 626, and a shaft 640 defining lumen extending through the shaft 640. The shaft 640 connects with a proximal end of an elongate body 603m of the cartridge 600m such that an opening 630m in the proximal end of the elongate body aligns with the lumen of the shaft 640. In use, the rod portion 634 of the deployment element 626 extends through the opening 630 in the proximal end of the elongate body 603m, along the lumen of the shaft 634, and extends proximally beyond the shaft 640 as shown. In some embodiments, the system also includes a handle affixed to or integral with a proximal end of the shaft 642 and the rod portion 634 of the deployment element extends proximally beyond the handle 642. The shaft 642 is used to control a position of the cartridge 600m while the deployment element 626 is axially displaced relative to the elongate body 603m to deploy the mesh 606 from the cavity.

FIG. 11A schematically depicts a side cross-sectional view of a surgical tool (e.g., a laparoscopic assist tool 618) pushing a cartridge 600 through a trocar 50 in accordance with some embodiments. Turning to FIG. 11B, in some embodiments, a different surgical tool 619 (e.g., a different laparoscopic assist tool) may be used to exert force on a mesh removal element (e.g., a tab of a surgical mesh 608) to remove the surgical mesh 606 from the cartridge 600. FIG. 11C depicts the laparoscopic assist tool 619 being used to pull on the tab 608 of the surgical mesh 606 to remove the surgical mesh 606 from the cartridge 600 after the cartridge is disposed beyond the trocar 52 in the internal body cavity 700. In some embodiments, an end effector of a robotic arm may be used to hold an elongate body during removal of the surgical mesh 608 from the cartridge. In some embodiments, an end effector of a robotic arm may be used to exert force on a mesh removal element, e.g., a tab 608.

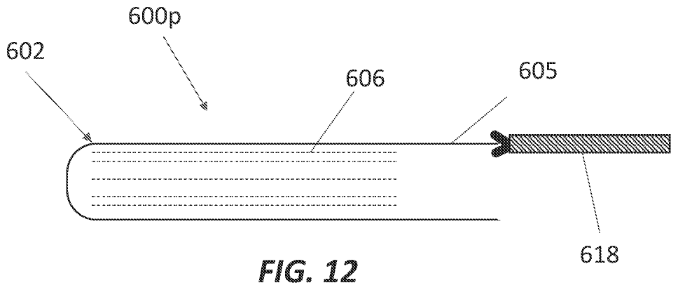

FIG. 12 schematically depicts a side view of inserting a cartridge 600p with the assistance of a sleeve 605 in accordance with some embodiments.

Described herein is a method of delivering at least one surgical mesh 606 to an internal body cavity of a subject during a surgical procedure. The method includes delivering a cartridge (e.g., cartridge 600) into the internal body cavity via a channel. Delivering the cartridge 600 may include grasping the elongate body 603 of the cartridge 600 with a surgical tool (e.g., surgical tool 618). For example, the surgical tool 618 may grasp the elongate body 603 at one or more engagement elements 610.

The method further includes retrieving at least one surgical mesh 606 from a cavity 601 of the cartridge 600 through the leading end 602 or the trailing end 604 of the elongate body 603 or deploying the at least one surgical mesh 606 from the cavity 601 via the leading end 602 or the trailing end 604 of the elongate body 603.

In some embodiments, the at least one surgical mesh 606 is deployed from the cavity 601 via the leading end 602. In some embodiments, deploying the surgical mesh 606 from the cavity 101 may include exerting a force on a deployment element 626 at least partially disposed within the cavity. See examples described above with respect to FIGS. 10C and 10D. Deploying the at least one surgical mesh 606 from the cavity 601 via the leading end 602 may include displacing a proximal end of a deployment element 620 relative to the elongate body 603, the deployment element 626 including a distal end 632 disposed in the cavity 601 of the elongate body 603 and including a rod shaped portion 634 extending out of the trailing end 602 of the elongate body 603. The displacement of the proximal end of the deployment element 626 relative to the elongate body 603 pushing the surgical mesh 606 out of the leading end 602 of the elongate body 603.

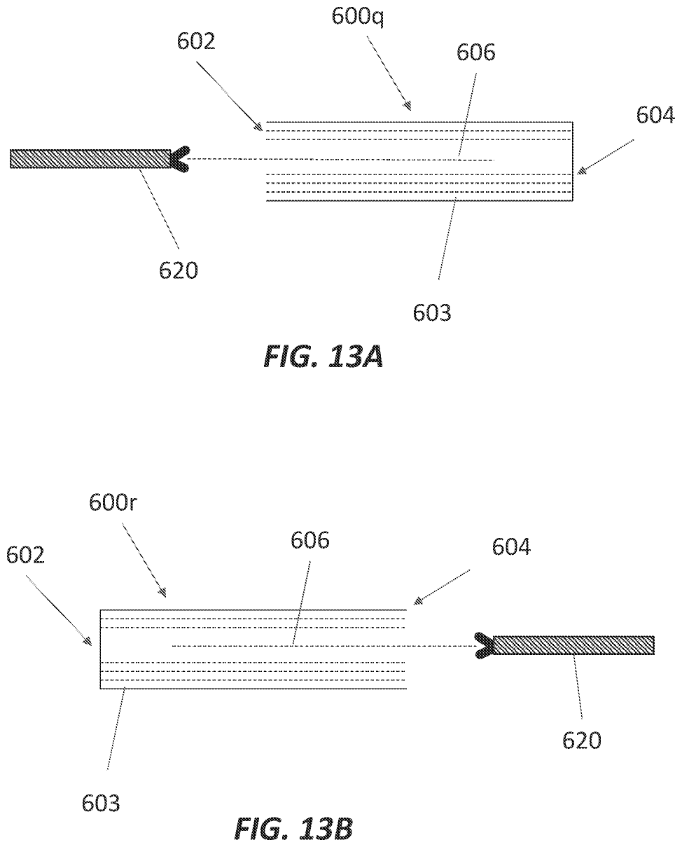

FIGS. 13A and 13B schematically depict retrieval of the surgical mesh 606 from the elongate body 603 in accordance with some embodiment. In FIG. 13A a surgical mesh 606 is retrieved from a leading end 602 of a cartridge 600q with the assistance of a surgical tool 620 in accordance with some embodiments. In FIG. 13B the surgical mesh 606 is retrieved from a trailing end 604 of a cartridge 600q in accordance with some embodiments. Retrieving the at least one surgical mesh 606 may include employing a surgical tool 620 or an end effector of a robotic arm to withdraw the at least one surgical mesh 606 from the cavity 601.

Retrieving the at least one surgical mesh 606 from the cavity 601 through the leading end 602 or the trailing end 604 of the elongate body 603 may include exerting a force on a mesh removal element 620, 622 that is at least partially disposed within the cavity 601 such that withdrawal of the mesh removal element 620, 622 from the cavity 601 initiates removal of the at least one surgical mesh 606 from the cavity 601. Retrieving the at least one surgical mesh 606 may include employing an end effector of a robotic arm to withdraw the at least one surgical mesh 606 from the cavity 601.

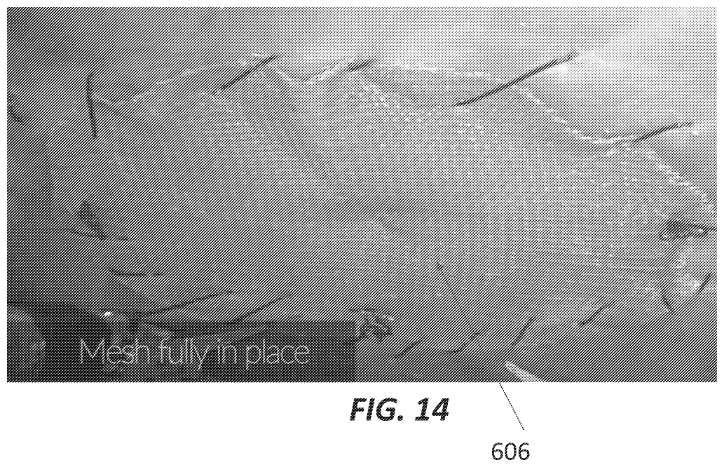

FIG. 14 is an image of a surgical mesh 606 unrolled and affixed to tissue within a subject's (e.g., patient's) abdomen during an IPOM procedure in accordance with some embodiments.

Further, the present disclosure provides systems, devices, and methods for assisted surgical mesh placement for surgical robotics for repairing anatomical defects. For example, hernia repair is a procedure that includes closing a wound or defect in tissue, such as muscle, of a subject. A common surgical repair technique may employ a surgical mesh placed over the hernia to reinforce the tissues surrounding the wound or defect. Surgical meshes may be made of a polymeric material, or a biologic or dissolvable material. Due to variability in the ways surgeons place meshes, there may be significant variability in patient outcomes. Systems, devices, and methods of the present disclosure may use sensors embedded on a surgical robotic system to help increase consistency of anatomical repair procedures (e.g., hernia procedures), and may be combined with use of a comprehensive database that includes specific measurable parameters as well as patient outcomes.

Systems, devices, and methods for assisted mesh placement for surgical robotics may advantageously bring together and analyze data that is available to surgical robotics platforms with patient outcomes to improve the outcomes of hernia repair procedures. Further, the system may be intelligent and adaptable to different conditions, while still allowing increased consistency of procedures performed.

In another aspect, the present disclosure provides a surgical robotic system comprising a set of sensors embedded thereon, wherein the surgical robotic system is configured to perform a surgical hernia repair procedure with increased consistency.

In another aspect, the present disclosure provides a method for using a set of sensors embedded on a surgical robotic system to perform a surgical anatomical repair procedure, e.g., a hernia procedure, with increased consistency.

Mesh fixation tension may be used as follows. A surgical robotic system may be configured to measure or estimate the force that it is exerting at its distal end and display information regarding the measured or estimated force to a user or operator, thereby enabling monitoring of the amount of force exerted while throwing sutures during fixation of the mesh. This may enable a surgeon to properly tension the mesh during fixation and may result in better patient outcomes. Disclosure regarding force measuring and sensing using surgical robotic systems may be found, at least, in International Patent Application No. PCT/US2022/29231 filed May 13, 2022. The system may be further configured to record the force data through the entire procedure and add the recorded force data to a database of other procedures. The database may also contain patient outcomes and, utilizing statistical or artificial intelligence (AI) techniques, the system may calculate forces that result in better patient outcomes. In some embodiments, the calculated force may be utilized as a warning threshold for the surgeon, such as by alerting the surgeon when they are outside of optimal force targets, thereby increasing the likelihood that the amount of force applied to the sutures and hernias is optimal for positive patient outcomes. In some embodiments, the calculated force may then be utilized intraoperatively by an intelligent surgical system to adjust the tension to the amount that reduces recurrence of hernia, pain, or other complications.

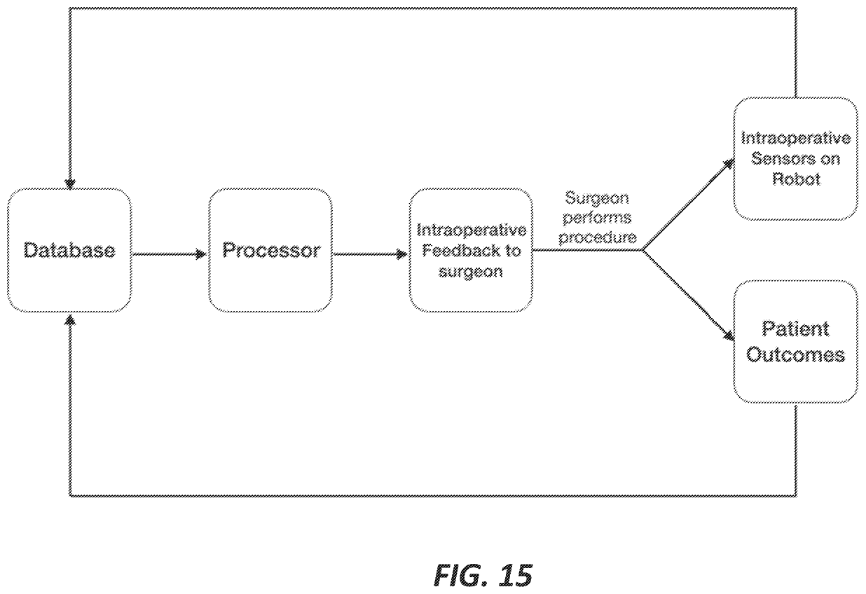

Mesh overlap may be used as follows. A surgical robotic system may be configured to measure intraoperative distances within the patient cavity, which can be utilized to properly size the overlap of the mesh with the hernia defect. Accordingly, described herein is a method of selecting a surgical mesh and delivering the surgical mesh to an internal body cavity during a surgical procedure. The method includes measuring at least one dimension of an anatomical defect within the internal body cavity during the surgical procedure. In some embodiments, the measurement may be by placing an end effector of a first robotic arm at a first position relative to the anatomical defect and an end effector of a second robotic at a second position relative to the anatomical defect, and the surgical robotic system using the location data regarding the first and second positions to determine at least one measurement of an extent of the anatomical defect. In some embodiments, an extent of an anatomical defect may be obtained via imaging. FIG. 15 schematically depicts a process for guiding a surgeon with respect to a surgical procedure. Once the size of the anatomical defect (e.g., hernia) is known by the system, the system may then utilize a database containing hernia size, mesh size, and/or overlap to determine a desired mesh size for the hernia that minimizes recurrence. The system may determine this desired mesh size utilizing statistical or artificial intelligence techniques. In some embodiments, the system is configured to guide the surgeon to select the correct mesh within a plurality of choices (e.g., within a specific vendor or catalog) of pre-filled cartridges. For example, the surgical robotic system may display information regarding the determined cartridge to be selected via a user interface. Such intelligent selection of meshes may enable the system (e.g., guided by a physician) to perform analyses on the data to continuously evaluate the outcomes of patients for a given set of parameters, and to adjust the algorithm over time to account for changes in patient outcomes. For example, the system may be configured to monitor patient outcomes on each side of the cutoff between two mesh sizes and determine or predict the outcome difference, such as via a regression discontinuity study design.

In some embodiments, the surgical robotic system may determine one or more dimensions of a surgical mesh to employed based on the at least one measurement of the anatomical defect in some embodiments. The determination of one or more dimensions may employ a look up table in some embodiments. The surgical robotic system may display the determined one or more dimensions of a surgical mesh to employed to a user in some embodiments. In some embodiments, surgical robotic system may alternatively or additionally display an identification of a cartridge holding a surgical mesh having the desired dimensions. During the surgical procedure, based on the displayed information regarding the determined one or more dimensions or regarding an identification of a cartridge (e.g., cartridge 600) holding a surgical mesh having the desired dimensions, a medical professional (e.g., a surgeon) may select a pre-filled cartridge holding a surgical mesh having the desired dimensions from a plurality of pre-filed cartridges, each holding a surgical mesh with different dimensions for use in the surgical procedure. Each cartridge in the plurality of cartridges may hold a surgical mesh having a different length, width, or diameter than that held by other cartridges in the plurality of cartridges. The selected cartridge is then delivered to the internal body cavity during the surgical procedure. Then the at least one surgical mesh is retrieved from the cavity through the leading end or the trailing end of the elongate body or is deployed from the leading end or the trailing end of the elongate body.

In some embodiments, the intraoperative data is continuously sent and stored in the database along with patient outcomes. In some embodiments, the database is static or maintained manually with curated data from specific physicians and scientists.

In some embodiments, computer vision techniques can further be intraoperatively utilized to assess proper mesh placement and/or to guide the surgeon to properly place and overlap the hernia.

While preferred embodiments of the present invention have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

1. A cartridge for storing and delivering a surgical mesh to an internal body cavity during a surgical procedure, the cartridge comprising:
an elongate body defining a cavity and having a first end configured to be a leading end during delivery of the cartridge to the internal body cavity and a second end configured to be a trailing end during delivery of the cartridge to the internal body cavity, one or both of the leading end and the trailing end having an opening therein, a cross-sectional, width, height or diameter of the elongate body being suitable for delivery during the surgical procedure; and
at least one surgical mesh at least partially disposed within the cavity.

2. The cartridge of claim 1, wherein the leading end is tapered.

3. The cartridge of claim 1, wherein the leading end is rounded.

4. The cartridge of claim 1, wherein the elongate body comprises at least one engagement element on an outer surface or as a portion of the outer surface of the elongate body disposed proximate to the trailing end.

5. The cartridge of claim 4, wherein the at least one engagement element is one of textured surface, a protuberance, a handle, or a shape configured to facilitate engagement with a surgical tool.

6. The cartridge of claim 1, wherein the surgical mesh is rolled, folded, or rolled and folded when disposed at least partially within the cavity.

7. The cartridge of claim 1, further comprising a mesh removal element at least partially disposed within the cavity such that withdrawal of the mesh removal element from the cavity initiates removal of the surgical mesh from the cavity.

8. The cartridge of claim 7, wherein the mesh removal element comprises one or more of: a tab, a string, or an elongate strip.

9. The cartridge of claim 7, wherein the mesh removal element is affixed to or is a part of the at least one surgical mesh.

10. The cartridge of claim 7, wherein the mesh removal element extends from the leading end of the elongate body.

11. The cartridge of claim 7, wherein the mesh removal element extends from the trailing end of the elongate body.

12-15. (canceled)

16. A method of delivering at least one surgical mesh to an internal body cavity of a subject during a surgical procedure, the method comprising:
delivering a cartridge into the internal body cavity via a channel during the surgical procedure, the cartridge comprising:
an elongate body defining a cavity and having a first end that is a leading end during delivery via the channel and a second end that is a trailing end during delivery via the channel; and
the at least one surgical mesh at least partially disposed within the cavity; and
retrieving the at least one surgical mesh from the cavity through the leading end or the trailing end of the elongate body or deploying the at least one surgical mesh from the cavity via the leading end or the trailing end of the elongate body.

17. The method of claim 16, wherein delivering the cartridge comprises grasping the elongate body with a surgical tool.

18. The method of claim 17, wherein the elongate body comprises at least one engagement element on an outer surface of the elongate body disposed proximate to the trailing end and wherein a surgical tool grasps the elongate body at the at least one engagement element.

19. The method of claim 18, wherein the at least one engagement element is one of textured surface, a protuberance, a handle, or a shape configured to facilitate engagement with the surgical tool.

20. The method of claim 16, wherein retrieving the at least one surgical mesh from the cavity through the leading end or the trailing end of the elongate body comprises exerting a force on a mesh removal element that is at least partially disposed within the cavity such that withdrawal of the mesh removal element from the cavity initiates removal of the at least one surgical mesh from the cavity.

21. The method of claim 20, wherein the mesh removal element is connected to, affixed to, or integral with the at least one surgical mesh.

22. The method of claim 20, wherein the mesh removal element comprises one or more of: a tab, a string, or an elongate strip.

23. The method of claim 16, wherein retrieving the at least one surgical mesh comprises withdrawing the at least one surgical mesh from the trailing end of the elongate body.

24. The method of claim 16, wherein retrieving the at least one surgical mesh comprises withdrawing the at least one surgical mesh from the leading end of the elongate body.

25. The method of claim 16, wherein retrieving the at least one surgical mesh comprises employing an end effector of a robotic arm to withdraw the at least one surgical mesh from the cavity.

26. The method of claim 16, wherein the at least one surgical mesh is deployed from the cavity via the leading end.

27. The method of claim 26, wherein deploying the at least one surgical mesh from the cavity via the leading end comprises displacing a proximal end of a deployment element relative to the elongate body, the deployment element including a distal end disposed in the cavity of the elongate body and including an elongate portion extending out of the trailing end of the elongate body, the displacement of the proximal end of the deployment element relative to the elongate body pushing the surgical mesh out of the leading end of the elongate body.

28.-34. (canceled)
